(19) United States
(12) Patent Application Publication
(10) Pub. No.: US 2009/0285759 A1
(43) Pub. Date: Nov. 19, 2009

(54) DRUG, DRUG GUIDANCE SYSTEM, MAGNETIC DETECTION SYSTEM, AND DRUG DESIGN METHOD

(76) Inventors: Yoshihiro Ishikawa, Tokyo (JP); Haruki Eguchi, Tokyo (JP)

Correspondence Address: OSTROLENK FABER GERB & SOFFEN, 1180 AVENUE OF THE AMERICAS, NEW YORK, NY, 100368403, US

(21) Appl. No.: 12/063,768

(22) PCT Filed: Aug. 23, 2006
(86) PCT No.: PCT/JP2006/317027

§ 371 Date: Feb. 14, 2008

(30) Foreign Application Priority Data
Aug. 31, 2005 (JP) 2005-251190
Jun. 28, 2006 (JP) 2006-177971

(51) Int. Cl. A61B 5/055; A61K 31/352; A61K 33/24

(52) U.S. Cl. 424/9.3; 514/455; 424/649

(57) ABSTRACT

A drug comprises an organic or inorganic compound, and is made magnetic by modification of side chains and/or crosslinking between side chains.

### BACKGROUND OF THE INVENTION

1. Field of the Invention

The present invention relates to a drug, a drug guidance system, a magnetic detection system, and a drug design method.

Priority is claimed on Japanese Patent Applications No. 2005-251190, filed Aug. 31, 2005, and No. 2006-177971, filed Jun. 28, 2006, the contents of which are incorporated herein by reference.

2. Description of the Related Art

Generally, drugs administered to the living body reach target sites and cause therapeutic effects by exerting pharmacological effects at the localized target sites. However, there will not be a cure if drugs reach tissues other than the target sites (that is, normal tissues). Consequently, how to guide drugs to the target sites efficiently is important in terms of therapeutic strategy. Such a technology for guiding drugs to the target sites is called drug delivery and research and development thereof have been actively carried out in recent years. These drug delivery methods have at least two merits. One is that a sufficiently high drug concentration is obtained in diseased tissues. This is advantageous because pharmacological effects are achieved only when the drug concentration in the target site is higher than a certain value, and therapeutic effects can not be expected when the concentration is low. Second is that the drug delivery methods guide drugs to diseased tissues only and do not guide drugs to normal tissues unnecessarily. Side effects can thereby be suppressed.

Such drug delivery methods exert their effects most in cancer treatments using anticancer agents. Since most anticancer agents suppress cell growth of cancer cells which are actively dividing, they also suppress cell growth in normal tissues where cells are actively dividing such as, for example, bone marrow, hair-roots, or gastrointestinal mucosa. On this account, side effects such as anemia, hair loss, and vomiting appear in cancer patients who have received administration of anticancer agents. Dosage has to be restricted since these side effects would be heavy burdens on patients and thus, there is a problem in that pharmacological effects of anticancer agents cannot be obtained sufficiently. Furthermore, there is a concern of patients dying due to the side effects in worst cases. Accordingly, it is hoped that cancer treatments can be carried out efficiently while suppressing the side effects by guiding the anticancer agents until they reach cancer cells with drug delivery methods and allowing the agents to exert their pharmacological effects on cancer cells specifically.

Apart from anticancer agents, for example, application of the drug delivery methods to agents for treating male erectile dysfunction is considered. There are examples of significant systemic hypotension resulting in deaths caused by the use of agents for treating male erectile dysfunction when combined with nitro preparations and thus, it is a problem particularly for males of middle and old age with heart disease. This is because the agents for treating erectile dysfunction do not necessarily concentrate at the target site, act on systemic blood vessels, and thereby increase vasodilation effects of nitro preparations. Accordingly, it is considered that the side effects resulting from the combined use with nitro preparations can be suppressed by guiding the agents for treating male erectile dysfunction until they reach the target site with drug delivery methods and allowing the agents to exert their pharmacological effects on the target site specifically.

As a specific method of drug delivery methods, for example, guidance to the target site using supports (carriers) is being studied and this method is to load drugs onto supports that tend to concentrate in the target site and thereby make the supports transport the drugs to the target site. As supports, use of various types of antibodies, microspheres, or magnetic bodies has been discussed. Among them, magnetic bodies are those that are regarded as particularly hopeful and a method to attach the supports, which are magnetic bodies, to the drugs and make them accumulate in the target site by means of a magnetic field has been examined (for example, refer to the following Patent Document 1). Since this guiding method is easy and simple and makes treatment which targets the target site possible, it is considered to be an effective method especially for anticancer agents with high cytotoxicity.

### Patent Document 1 Japanese Laid-Open Patent Application No. 2001-10978

However, when the supports, which are magnetic bodies, are used as carriers as described above, difficulties in oral administration, the large size of carrier molecules in general, or technical problems in bond strength and affinity with the drug molecules have been pointed out and thus, practical application has been difficult.

### SUMMARY OF THE INVENTION

The present invention addresses the abovementioned problems, with an object of realizing a drug delivery system which is capable of solving conventional technical problems and which is easy to put into practical application.

In order to achieve the above object, the present invention, as a first aspect according to a drug, comprises an organic or inorganic compound, and is made magnetic by modification of side chains and/or crosslinking between side chains.

Moreover, in the present invention, as a second aspect according to a drug, in the first aspect, the organic compound is forskolin.

Moreover, in the present invention, as a third aspect according to a drug, in the first aspect, the organic compound is a composition effective in the treatment of male erectile dysfunction.

Moreover, in the present invention, as a fourth aspect according to a drug, in the first aspect, the inorganic compound is a metal complex.

Moreover, in the present invention, as a fifth aspect according to a drug, in the fourth aspect, the metal complex is a cis geometric isomer with anticancer properties.

Moreover, in the present invention, as a sixth aspect according to a drug, in the fifth aspect, the cis geometric isomer is cisplatin.

Moreover, in the present invention, as a first aspect according to a drug guidance system, a drug of any one of the above first to sixth aspects administered to a body is guided to a predetermined target site using the magnetism of the drug.

Moreover, in the present invention, as a first aspect according to a magnetic detection system, by detecting magnetism of a drug of any one of the above first to sixth aspects administered in a body, the dynamics of the drug are detected.

Moreover, the present invention, as a first aspect according to a drug design method, comprises: setting with respect to an organic or inorganic compound used as a drug, a molecular model having modified side chains and/or crosslinked side chains; determining whether or not the molecular model is magnetic, from a spin-charge density distribution obtained by a numerical calculation for the molecular model; and then designing the organic compound based on the molecular model that has been determined to be magnetic.

Moreover, the present invention, as a second aspect according to a drug design method, comprises in the first aspect, determining whether the molecular model is ferromagnetic or ferrimagnetic, based on the spin-charge density distribution.

Moreover, the present invention, as a third aspect according to a drug design method, comprises in the first aspect, determining the magnetic strength of the molecular model, based on the spin-charge density distribution.

According to the present invention, since drugs themselves will be magnetic, it is possible to guide the drugs to the target sites in the body by use of magnetism of the drugs themselves without using supports made from magnetic bodies as in the conventional cases. As a result, conventional problems such as difficulties in oral administration, the large size of carrier molecules in general, or technical problems in bond strength and affinity with the drug molecules can be resolved. Furthermore, it is possible to realize a drug delivery system which is easy to put into practical application.

### BRIEF DESCRIPTION OF THE DRAWINGS

FIG. 1 is a diagram of a basic molecular structural model of forskolin in one embodiment of the present invention.

FIG. 2 is a diagram of a molecular structural model of a ferrimagnetic forskolin derivative A in one embodiment of the present invention.

FIG. 3 is a diagram showing a three-dimensional molecular structural model of the forskolin derivative A and its spin-charge density distribution, in one embodiment of the present invention.

FIG. 4 is a diagram of a molecular structural model of a ferromagnetic forskolin derivative B in one embodiment of the present invention.

FIG. 5 is a diagram showing a three-dimensional molecular structural model of the forskolin derivative B and its spin-charge density distribution, in one embodiment of the present invention.

FIG. 6 is a flow chart of a drug design method in one embodiment of the present invention.

FIG. 7A is a diagram of a basic molecular structural model of PDE5 inhibitor with a standard composition and FIG. 7B shows a three-dimensional molecular structure and spin-charge density distribution of the PDE 5 inhibitor with a standard composition in one embodiment of the present invention.

FIG. 8A is a diagram of a basic molecular structural model of a derivative of PDE5 inhibitor and FIG. 8B shows a three-dimensional molecular structural model and spin-charge density distribution of the derivative of PDE 5 inhibitor in one embodiment of the present invention.

FIG. 9 is a diagram of a basic molecular structural model of cisplatin in one embodiment of the present invention.

FIG. 10A is a diagram of a basic molecular structural model of a cisplatin derivative (Cis-Pt-a3) and FIG. 10B shows a three-dimensional molecular structural model and spin-charge density distribution of the cisplatin derivative (Cis-Pt-a3) in one embodiment of the present invention.

FIG. 11 is an analytical result of spin-charge densities of a cisplatin derivative and a derivative derived by the substitution of platinum of the cisplatin derivative into another metal element in one embodiment of the present invention.

FIG. 12 is a diagram of a basic molecular structural model of the cisplatin derivative NK121, and its three-dimensional molecular structural model and spin-charge density distribution in one embodiment of the present invention.

FIG. 13 is a diagram showing a hydrolysis process of cisplatin in a living body in one embodiment of the present invention.

FIG. 14 is a diagram of a three-dimensional molecular structural model and spin-charge density distribution of the cisplatin hydrolysate [Pt(OH₂)₂(dien)]²⁺ in one embodiment of the present invention.

### DETAILED DESCRIPTION OF THE INVENTION

Hereunder is a description of one embodiment of the present invention, with reference to the drawings.

### First Embodiment

Firstly, the first embodiment is described using an organic compound, more specifically, forskolin, as a drug candidate agent.

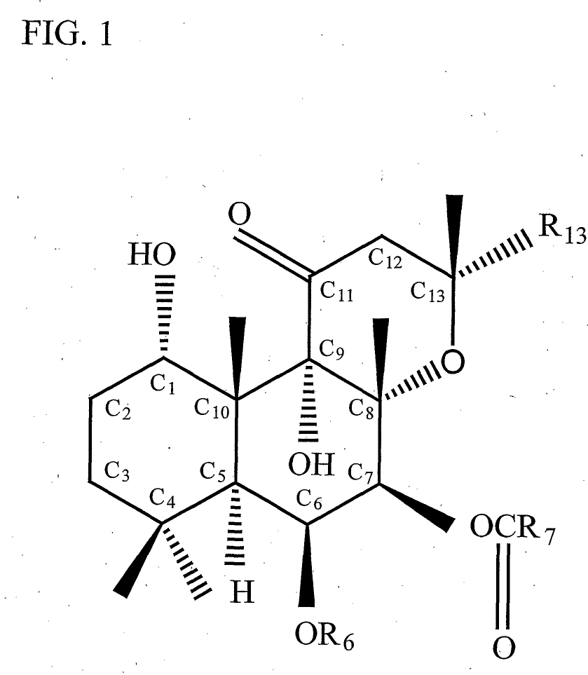

FIG. 1 is a diagram showing a basic molecular structural model of forskolin. In this drawing, R₆, R₇, and R₁₃ show positions bonded with an atom or a molecule for modifying a side chain of forskolin. Depending on the type of atom or molecule bonded to these positions, the physical property of forskolin varies. In this drawing, one having H bonded to R₆, CH₃ bonded to R₇, and CH═CH₂ bonded to R₁₃ is naturally occurring forskolin, and one having the side chain structure changed artificially, that is, forskolin produced by changing the atom or molecule for modifying R₆, R₇, and R₁₃, is called a forskolin derivative. In FIG. 1, C₁ to C₁₃ represent a carbon atom (C).

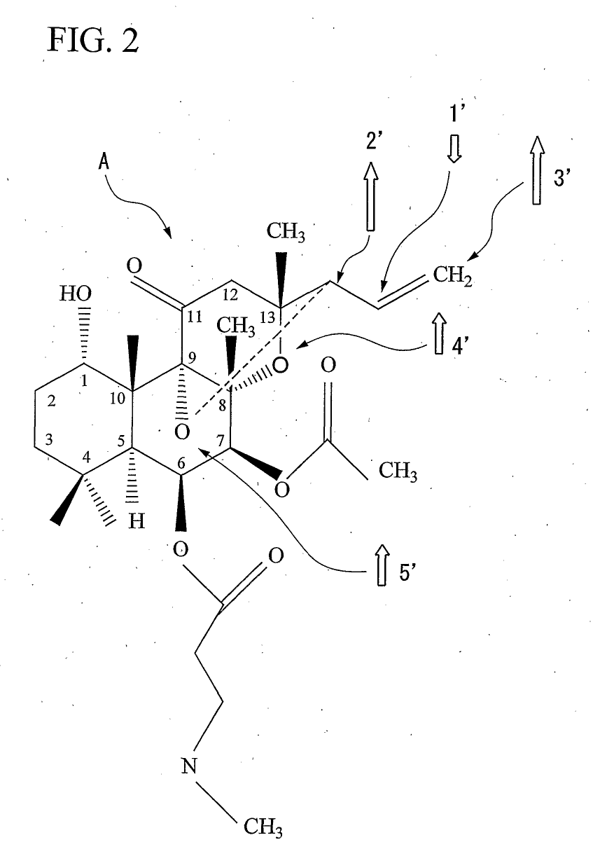

FIG. 2 is a diagram showing a basic molecular structural model of a magnetic (ferrimagnetic) forskolin derivative A. As shown in this drawing, the forskolin derivative A is one where R₆ of the abovementioned naturally occurring forskolin is changed into COCH₂CH₂NCH₃, R₇ is CH₃, R₁₃ is changed into CH—CH═CH₂, and the oxygen atom (O) bonded to C₉ and the carbon atom bonded to C₁₃ are crosslinked.

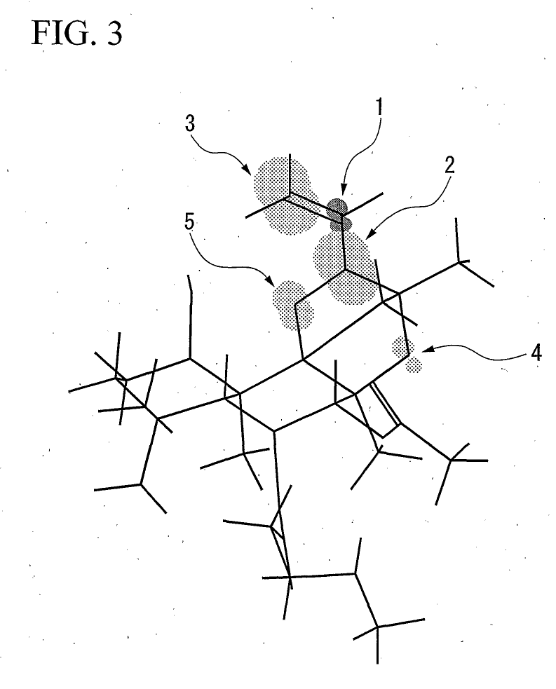

FIG. 3 shows a three-dimensional molecular structure of the forskolin derivative A and its spin-charge density distribution obtained by a computer simulation based on a well-known first principle molecular dynamics method. In FIG. 3, a region 1 shows a downward spin-charge density, and regions 2 to 5 show upward spin-charge densities. Therefore, as shown in FIG. 2, since a downward spin state 1′ and upward spin states 2′ to 5′ are mixed in the forskolin derivative A, it is found to be a ferrimagnetic body.

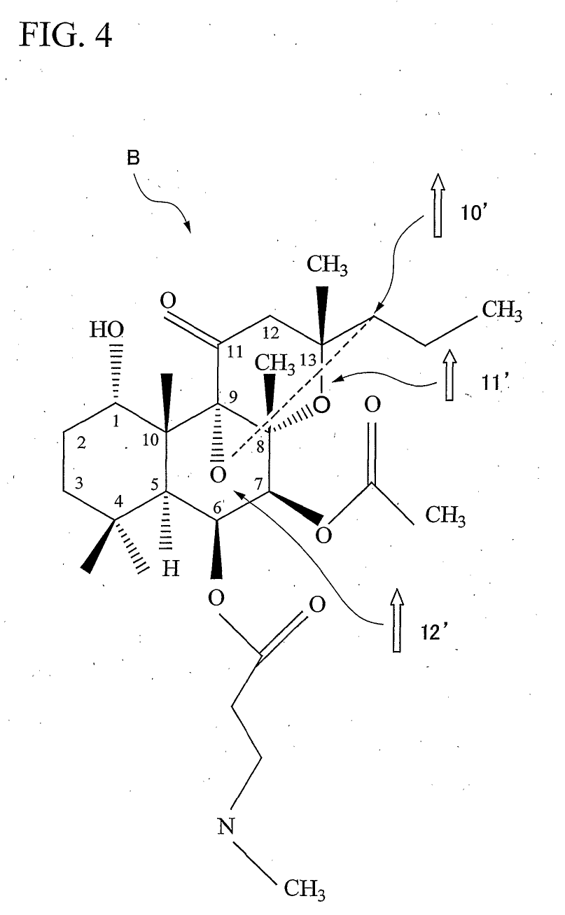

On the other hand, FIG. 4 is a diagram showing a basic molecular structural model of a magnetic (ferromagnetic) forskolin derivative B. As shown in this drawing, the forskolin derivative B is one where R₆ of the abovementioned naturally occurring forskolin is changed into COCH₂CH₂NCH₃, R₇ is CH₃, R₁₃ is changed into CH—CH₂—CH₃, and the oxygen atom bonded to C₉ and the carbon atom bonded to C₁₃ are crosslinked.

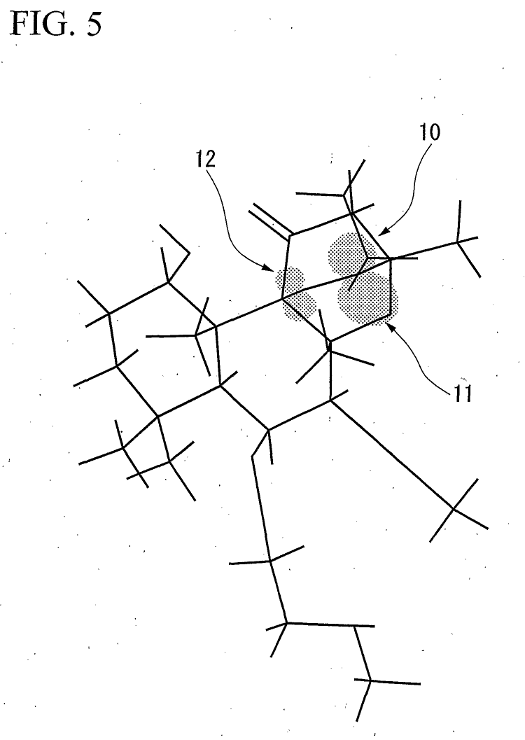

Similarly to the above, FIG. 5 shows a three-dimensional molecular structure of the forskolin derivative B and its spin-charge density distribution obtained by a computer simulation based on the first, principle molecular dynamics method. In FIG. 5, regions 10 to 12 show upward spin-charge densities. Therefore, as shown in FIG. 4, since only upward spin states 10′ to 12′ are present in the forskolin derivative B, it is found to be a ferromagnetic body.

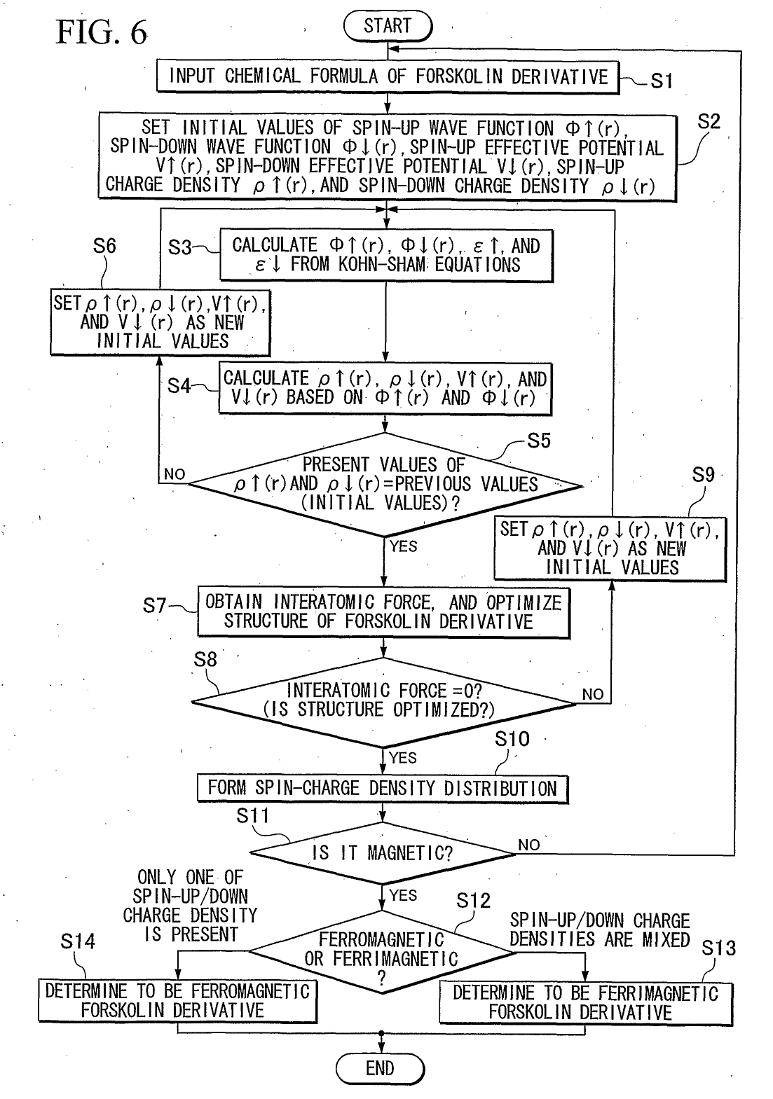

In this manner, by modifying the side chains of forskolin with predetermined atoms or molecules, and crosslinking between side chains present in predetermined positions, a magnetic forskolin derivative, that is, a drug, can be produced. Hereunder is a description of a design method for such a magnetic drug. FIG. 6 is a flow chart showing a processing procedure of the present drug design method. The processing described hereunder is performed in a computer simulation based on the first principle molecular dynamics method.

Firstly, since there are more than 200 types of forskolin derivatives used as drugs, a forskolin derivative serving as an evaluation target is selected from among these, and its chemical formula is input into the computer simulation (step S1). Here, a case where the abovementioned forskolin derivative A is selected as the forskolin derivative is assumed and described hereunder.

Subsequently, based on the chemical formula of the forskolin derivative A, initial values of upward spin (spin up) wave function Φ_{↑}(r), downward spin (spin down) wave function Φ_{↓}(r), spin-up effective potential V_{↑}(r), spin-down effective potential V_{↓}(r), spin-up charge density ρ_{↑}(r), and spin-down charge density ρ_{↓}(r) are set (step S2). Here r is a variable showing the coordinates in the three-dimensional space.

In a case where the respective atoms constituting the forskolin derivative A are present as an isolated atom in the three-dimensional space, the spin-up wave functions Φ_{↑}(r) are obtained for each of the respective atoms. The initial value of the spin-up wave function Φ_{↑}(r) is the sum of all the spin-up wave functions Φ_{↑}(r) that have been obtained in such a manner. Similarly, the initial value of the spin-down wave function Φ_{↓}(r) is the sum of all the spin-down wave functions Φ_{↓}(r) obtained for each of the respective atoms, in a case where the respective atoms are present as an isolated atom in the three-dimensional space. Moreover, based on the spin-up wave functions Φ_{↑}(r) in a case where the respective atoms constituting the forskolin derivative A are present as an isolated atom in the three-dimensional space, the spin-up effective potentials V_{↑}(r) are obtained for each of the respective atoms. The initial value of the spin-up effective potential V_{↑}(r) is the sum of all the spin-up effective potentials V_{↑}(r) that have been obtained for each of the respective atoms. Similarly, the initial value of the effective potential V_{↓}(r) is the sum of all the spin-down effective potentials V_{↓}(r) obtained for each of the respective atoms based on the spin-down wave functions Φ_{↓}(r) in a case where the respective atoms are present as an isolated atom in the three-dimensional space. Moreover, the initial value of the spin-up charge density ρ_{↑}(r) is obtained by substituting the spin-up wave functions Φ_{↑}(r) that have been obtained for each of the respective atoms as mentioned above, into the following operational expression (1). Moreover, the initial value of the spin-down charge density ρ_{↓}(r) is obtained by substituting the spin-down wave functions Φ_{↓}(r) that have been obtained for each of the respective atoms, into the following operational expression (2). In the following operational expression (1), Φ_{↑}*(r) is a conjugate complex number of the spin-up wave function Φ_{↑}(r). In the following operational expression (2), Φ_{↓}*(r) is a conjugate complex number of the spin-down wave function Φ_{↓}(r).

### [Equation 1]

ρ_{↑}(r)=ΣΦ_{↑}*(r)Φ_{↑}(r) (1)

ρ_{↓}(r)=ΣΦ_{↓}*(r)Φ_{↓}(r) (2)

Next, based on the initial values of the spin-up effective potential V_{↑}(r) and the spin-down effective potential V_{↓}(r), and the initial values of the spin-up charge density ρ_{↑}(r) and the spin-down charge density ρ_{↓}(r), the following Kohn-Sham equations (3) and (4) are solved, so as to calculate the spin-up wave function Φ_{↑}(r), the spin-down wave function Φ_{↓}(r), the spin-up energy eigenvalue ε_{↑}, and the spin-down energy eigenvalue ε_{↓} of the forskolin derivative A (step S3).

$$\begin{matrix}[ Equation 2 ] & \\ [ - \frac{1}{2} {∇}^{2} + {V}_{↑} \{ r , {ρ}_{↑} ( r ) \} ] {Φ}_{↑} ( r ) = {ɛ}_{↑} {Φ}_{↑} ( r ) & ( 3 ) \\ [ - \frac{1}{2} {∇}^{2} + {V}_{↓} \{ r , {ρ}_{↓} ( r ) \} ] {Φ}_{↓} ( r ) = {ɛ}_{↓} {Φ}_{↓} ( r ) & ( 4 )\end{matrix}$$

Then, based on the spin-up wave function Φ_{↑}(r) and the spin-down wave function Φ_{↓}(r) of the forskolin derivative A obtained in step S3, the spin-up charge density ρ_{↑}(r), the spin-down charge density ρ_{↓}(r), the spin-up effective potential V_{↑}(r), and the spin-down effective potential V_{↓}(r) of the forskolin derivative A are calculated (step S4). It is then determined whether or not these spin-up charge density ρ_{↑}(r) and spin-down charge density ρ_{↓}(r) are the same as the previous values of the spin-up charge density ρ_{↑}(r) and the spin-down charge density ρ_{↓}(r), which are the initial values in this case (step S5). In this step S5, if it is determined “NO”, that is, the previous values (initial values) of the spin-up charge density ρ_{↑}(r) and the spin-down charge density ρ_{↓}(r) are not the same as the present values obtained in step S4, then the spin-up effective potential V_{↑}(r), the spin-down effective potential V_{↓}(r), the spin-up charge density ρ_{↑}(r), and the spin-down charge density ρ_{↓}(r) obtained in step S4 are set as new initial values (step S6). Then the flow proceeds to step S3, and the Kohn-Sham equations (3) and (4) are solved again, so as to calculate a new spin-up wave function Φ_{↑}(r), spin-down wave function Φ_{↓}(r), spin-up energy eigenvalue ε_{↑}, and spin-down energy eigenvalue ε_{↓}. That is, in step S5, the processing from steps S3 to S6 is repeated until the previous values of the spin-up charge density ρ_{↑}(r) and the spin-down charge density ρ_{↓}(r) become equal to the present values, to thereby obtain the spin-up wave function Φ_{↑}(r), the spin-down wave function Φ_{↓}(r), the spin-up energy eigenvalue ε_{↑}, and the spin-down energy eigenvalue ε_{↓} which satisfy the Kohn-Sham equations (3) and (4).

On the other hand, in step S5, if it is determined “YES”, that is, the previous values of the spin-up charge density ρ_{↑}(r) and the spin-down charge density ρ_{↓}(r) are the same as the present values, then as described above, an interatomic force acting between respective atoms is calculated, based on the spin-up wave function Φ_{↑}(r), the spin-down wave function Φ_{↓}(r), the spin-up energy eigenvalue ε_{↑}, and the spin-down energy eigenvalue ε_{↓} which satisfy the Kohn-Sham equations (3) and (4), and the structure of the forskolin derivative A is optimized (step S7). That is, the spin-up wave function Φ_{↑}(r), the spin-down wave function Φ_{↓}(r), and so forth that have been obtained by repeating steps S3 to S6, are merely the optimum values in a model on a two-dimensional plane as shown in FIG. 2, and in practice it is necessary to consider the structure of the forskolin derivative A in the three-dimensional space.

Specifically, in step S7, the respective atoms constituting the forskolin derivative A are moved for a predetermined distance in an optimum direction assumed from the spin-up wave function Φ_{↑}(r) and the spin-down wave function Φ_{↓}(r), in the three-dimensional space, and an interatomic force acting between the respective atoms at this time is calculated. If the interatomic force at this time becomes 0 and the respective atoms are not moved, it can be determined that the structure of the forskolin derivative A is optimized. Therefore, the interatomic force acting between the respective atoms after the movement is calculated, and it is determined whether or not the interatomic force becomes 0 (step S8). In this step S8, if it is determined “NO”, that is, the interatomic force is not 0 and the structure is not optimized, then the spin-up wave functions Φ_{↑}(r) and the spin-down wave functions Φ_{↓}(r) in the structures of the respective atoms after the movement are obtained. Then, the spin-up effective potential V_{↑}(r), the spin-down effective potential V_{↓}(r), the spin-up charge density ρ_{↑}(r), and the spin-down charge density ρ_{↓}(r) obtained from the spin-up wave function Φ_{↑}(r) and the spin-down wave function Φ_{↓}(r) are set as new initial values (step S9), and the processing from steps S3 to S8 is repeated. Here, the reason the flow returns to step S3 is that the spin-up wave function Φ_{↑}(r) and the spin-down wave function Φ_{↓}(r) are changed according to the structural change of the respective atoms after the movement. Moreover, the structures of the respective atoms after the movement are memorized, and when step S7 is performed again, the respective atoms are moved again for a predetermined distance from the previous structure.

When the structure of such a forskolin derivative A is optimized, then as shown in FIG. 2, the three-dimensional structure is forcibly altered so as to crosslink the oxygen atom bonded to C₉ and the carbon atom bonded to C₁₃. The atoms selected for such a crosslinking can be optionally changed.

On the other hand, in this step S8, if it is determined “YES”, that is, the interatomic force acting between the respective atoms becomes 0 and the structure of the forskolin derivative A is optimized, then the spin-charge density distribution as shown in FIG. 3 is obtained, based on the spin-up wave function Φ_{↑}(r) and the spin-down wave function Φ_{↓}(r) in the optimized structure (step S10).

Here, depending on the forskolin derivative selected as the evaluation target, the spin-charge density distribution such as regions 1 to 5 shown in FIG. 3 is not generated, or if the spin-charge density distribution is generated, regions having only a very small amount of spin-charge density (that is magnetic strength) are present. Such a forskolin derivative can not be determined to be magnetic. Consequently, based on the spin-charge density distribution, firstly it is determined whether or not the forskolin derivative selected as the evaluation target is magnetic (step S11).

In step S11, if it is determined “NO”, that is, the forskolin derivative selected as the evaluation target is not magnetic, the flow proceeds to step S1, and another forskolin derivative is newly selected and the magnetism is evaluated again. On the other hand, in step S11, if it is determined “YES”, that is, the forskolin derivative selected as the evaluation target is magnetic, then it is determined whether it is ferromagnetic or ferrimagnetic, based on the spin-charge density distribution (step S12). As described above, since the spin-charge density distribution shows the distribution of the spin-up charge density and the spin-down charge density, if these spin-up charge density and spin-down charge density are mixed, it can be determined to be ferrimagnetic. If only one of the spin-up charge density and the spin-down charge density is present, it can be determined to be ferromagnetic.

As shown in FIG. 3, in the forskolin derivative A, since the spin-up charge densities (regions 2 to 5) and the spin-down charge density (region 1) are mixed, it is determined to be a ferrimagnetic forskolin derivative (step S13). On the other hand, for example, if the selected forskolin derivative is the forskolin derivative B, as shown in FIG. 5, only the spin-up charge densities (regions 10 to 12) are present. Therefore it is determined to be a ferromagnetic forskolin derivative (step S14). It is also possible to obtain the magnetic strength based on the spin-charge density distribution.

As described above, according to the present drug design method, the magnetism of a forskolin derivative having side chains modified with various atoms or molecules, and side chains optionally crosslinked can be determined. Moreover, by producing a forskolin derivative based on a molecular model determined to be magnetic, a magnetic drug can be manufactured. Therefore, it is possible to guide the drugs to the target sites in the body by use of magnetism of the drugs themselves without using supports (carriers) made from magnetic bodies as in the conventional cases. As a result, conventional problems such as difficulties in oral administration, the large size of carrier molecules in general, or technical problems in bond strength and affinity with the drug molecules can be resolved. Furthermore, it is possible to realize a drug delivery system which is easy to put into practical application.

In the above first embodiment, in both the forskolin derivatives A and B, the three-dimensional structure is forcibly altered so as to crosslink the oxygen atom bonded to C₉ and the carbon atom bonded to C₁₃. However, it is not limited to this, and other atoms may be selected to be crosslinked. Moreover, by not performing crosslinking, but by simply changing an atom or a molecule for modifying the side chain, it may be determined to be magnetic or not.

Moreover, in the above first embodiment, forskolin is used as an organic compound for description. However, it is not limited to this, and other organic compounds may be used. Hereunder is a description of, as another organic compound, a composition effective in treatments of male erectile dysfunction, more specifically, a composition inhibiting the generation of phosphodiesterase 5 (PDE 5), which hereinafter will be referred to as “PDE 5 inhibitor”. Drugs having this PDE 5 inhibitor as an active ingredient are used as remedies for male erectile dysfunction such as so-called Viagra®.

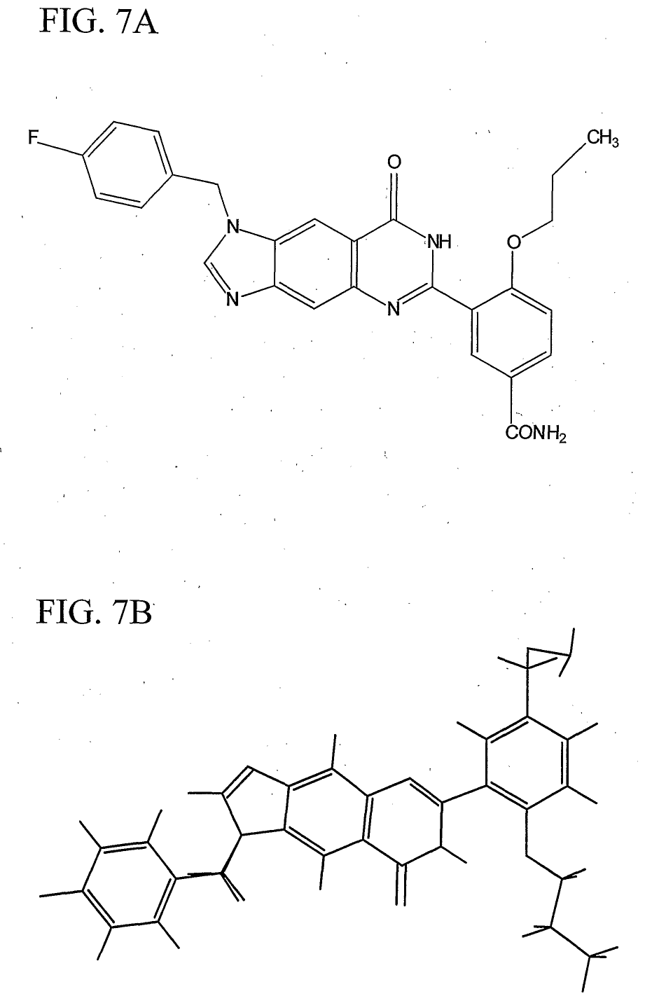

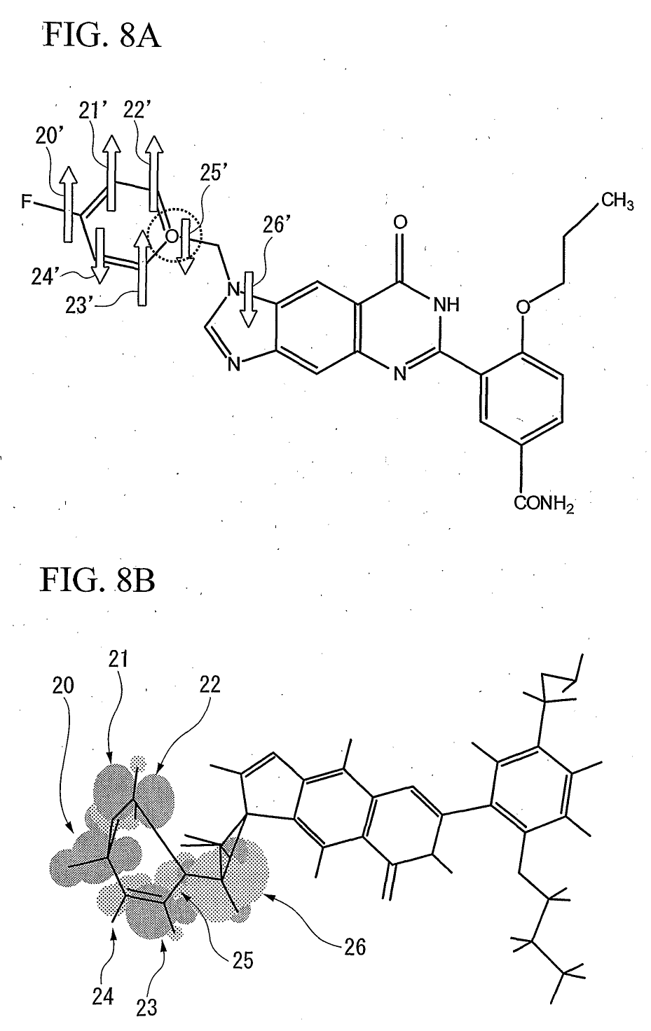

FIG. 7A is a diagram of a basic molecular structural model of PDE5 inhibitor with a standard composition and FIG. 7B shows a three-dimensional molecular structure and spin-charge density distribution of the PDE 5 inhibitor with a standard composition that are obtained by a computer simulation in the abovementioned drug design method. On the other hand, FIG. 8A is a diagram of a basic molecular structural model of a PDE 5 inhibitor derivative derived by subjecting the PDE 5 inhibitor with a standard composition to side chain modifications. FIG. 8B shows a three-dimensional molecular structure and spin-charge density distribution of the PDE 5 inhibitor derivative obtained by the abovementioned computer simulation. In FIG. 8B, the regions 20 to 23 show upward spin-charge densities, and the regions 24 to 26 show downward spin-charge densities. Therefore, the PDE 5 inhibitor derivative is a ferrimagnetic body where the upward spin states 20′ to 23′ and the downward spin states 24′ to 26′ coexist as shown in FIG. 8A.

That is, as shown in these FIGS. 7 and 8, although the PDE 5 inhibitor with a standard composition is not magnetic, the PDE 5 inhibitor derivative which is generated by side chain modification is confirmed to be magnetic. Therefore, by using a therapeutic agent for male erectile dysfunction, which has such a magnetic PDE 5 inhibitor derivative as an active ingredient, pharmacological effects of the drug can be brought out specifically in the target site and the occurrence of side effects due to the combined use with the nitro preparations can be suppressed.

### Second Embodiment

Next, a second embodiment is described using an inorganic compound, more specifically, cisplatin as an anticancer agent. Cisplatin is a metal complex (platinum complex) and classified as a platinum preparation among the anticancer agents.

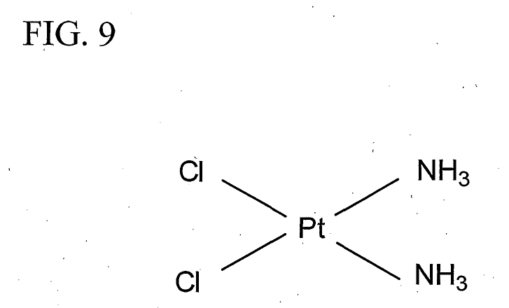

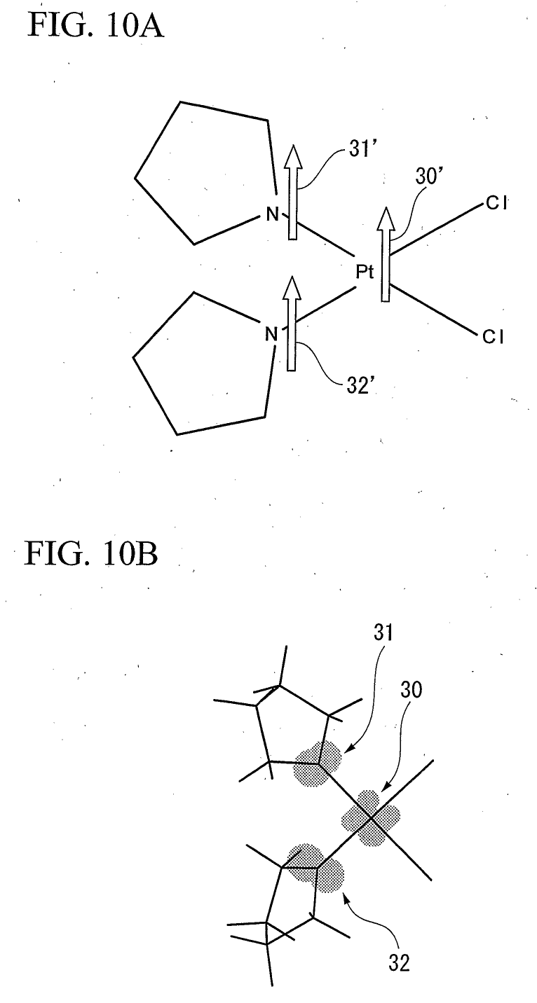

FIG. 9 is a diagram of a basic molecular structural model of cisplatin with a standard composition. Using the computer simulation in the drug design method described in the first embodiment, this cisplatin with a standard composition is confirmed to be non-magnetic. On the other hand, FIG. 10A is a diagram of a basic molecular structural model of a cisplatin derivative (Cis-Pt-a3), which is derived by subjecting the cisplatin with a standard composition to side chain modifications. Additionally, FIG. 10B shows a three-dimensional molecular structure and spin-charge density distribution of the cisplatin derivative (Cis-Pt-a3) obtained by the abovementioned computer simulation.

In FIG. 10B, the regions 30 to 32 show upward spin-charge densities. Therefore, the cisplatin derivative (Cis-Pt-a3) is found to be a ferromagnetic body where the upward spin states 30′ to 32′ exist as shown in FIG. 10A. That is, using the computer simulation in the present drug design method, the cisplatin derivative (Cis-Pt-a3) is confirmed to be magnetic. Therefore, by using an anticancer agent, which has such a magnetic cisplatin derivative (Cis-Pt-a3) as an active ingredient, pharmacological effects of the drug can be brought out specifically in the cancer tissues and the occurrence of side effects can be suppressed.

The stronger the magnetism of a drugs the more efficiently the drug can be guided to the target site, and thus, a greater increase in pharmacological effects and suppression of side effects can be expected. Accordingly, the present inventors carried out an analysis of magnetic strength for various cisplatin derivatives using the computer simulation in the present drug design method. The analytical results are described below. Since the magnetic strength is in a linear relationship with the spin-charge density, the spin-charge densities in various cisplatin derivatives are analyzed in the present embodiment.

Firstly, as a reference, particles having a total number of 101 atoms and which were approximately 8 Å on a side were cut out from a magnetite (Fe₃O₄) crystal and were set as the molecular models, and after electronic states and structures were optimized by the abovementioned computer simulation, the analysis of spin-charge densities was performed. Then, by adopting the spin-charge density of the abovementioned magnetite particles as the standard, the analysis of spin-charge densities for various cisplatin derivatives was similarly carried out.

Furthermore, in addition to the cisplatin derivatives, various derivatives where platinum (Pt) of the cisplatin derivatives was substituted by palladium (Pd), rhodium (Rh), iridium (Ir), gold (Au), nickel (Ni), silver (Ag), copper (Cu), or cobalt (Co) were similarly analyzed for their spin-charge densities. The derivatives generated by the substitution of platinum in the cisplatin derivatives with the abovementioned metal elements, as described above, are known to have effects in inhibiting the replication of DNA which is accompanied with the propagation of cancer cells, similarly to cisplatin or cisplatin derivatives.

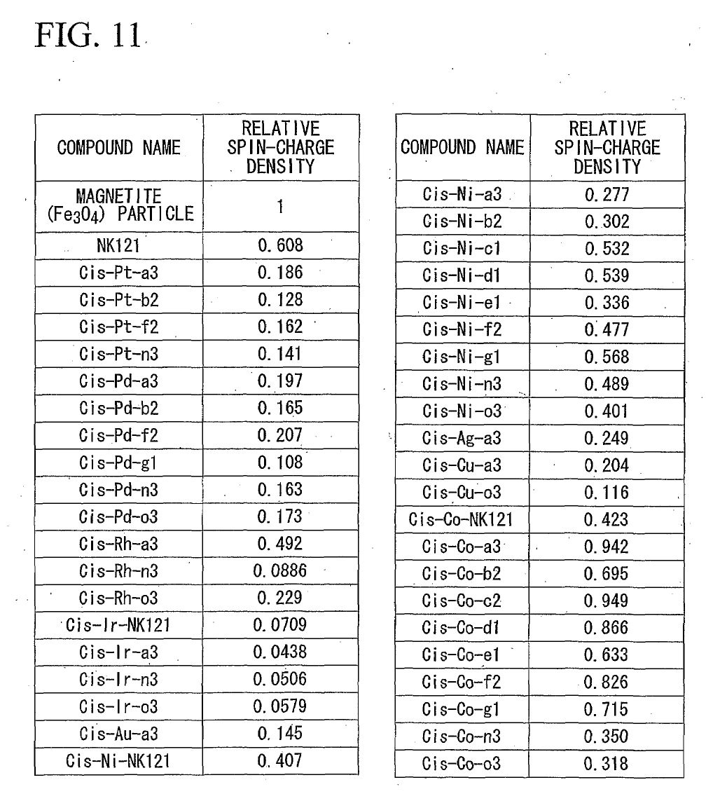

FIG. 11 shows the analytical results of spin-charge densities of various cisplatin derivatives and of various derivatives where platinum (Pt) of the cisplatin derivatives was substituted by palladium (Pd), rhodium (Rh), iridium (Ir), gold (Au), nickel (Ni), silver (Ag), copper (Cu), or cobalt (Co), when the spin-charge density of the magnetite particles was standardized to “1”.

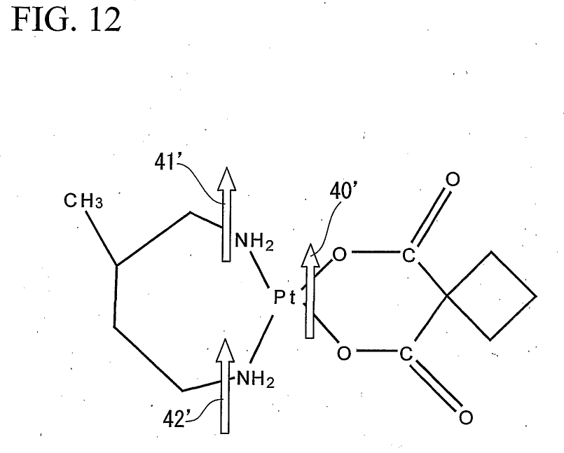

As shown in FIG. 11, among the cisplatin derivatives, it was found that NK121 had approximately 60% of spin-charge density compared to that of the magnetite particles and is effective as a magnetic drug compared to other cisplatin derivatives. This cisplatin derivative NK121 is one which once managed to reach clinical development after a safety test. However, since the anticancer effect thereof was comparable to that of cisplatin, it was determined to have no merits surpassing cisplatin and the development thereof was suspended. Therefore, if this cisplatin derivative NK121 is taken and the guidance of the drug to the target site by means of a magnetic field is performed, drug effects would increase and side effects can also be suppressed to a large extent. FIG. 12 shows a diagram of a basic molecular structural model of the cisplatin derivative NK121. As shown in this diagram, the cisplatin derivative NK121 is a ferromagnetic body where the upward spin states 40′ to 42′ exist.

Moreover, the derivatives where platinum (Pt) of the cisplatin derivatives was substituted by palladium (Pd) were also confirmed to have spin-charge densities to some extent and thus, were magnetic bodies. In addition, among the derivatives where platinum (Pt) of the cisplatin derivatives was substituted by rhodium (Rh), Cis-Rh-a3 was found to have approximately 50% of spin-charge density compared to that of the magnetite particles and was effective as a magnetic drug. Further, the derivatives where platinum (Pt) of the cisplatin derivatives was substituted by iridium (Ir) were confirmed to have considerably small spin-charge densities and not many effects as magnetic drugs. Additionally, the derivatives where platinum (Pt) of the cisplatin derivatives was substituted by gold (Au) were also confirmed to have spin-charge densities to some extent and were magnetic bodies.

Moreover, the derivatives where platinum (Pt) of the cisplatin derivatives was substituted by nickel (Ni) generally had approximately 50% of spin-charge densities compared to those of the magnetite particles and were found to be effective as magnetic drugs. Additionally, the derivatives where platinum (Pt) of the cisplatin derivatives was substituted by silver (Ag) were also confirmed to have spin-charge densities to some extent and were magnetic bodies. In addition, the derivatives where platinum (Pt) of the cisplatin derivatives was substituted by copper (Cu) were also confirmed to have spin-charge densities to some extent and were magnetic bodies. Furthermore, it was found that the derivatives where platinum (Pt) of the cisplatin derivatives was substituted by cobalt (Co) had, among higher ones thereof, approximately 95% of spin-charge densities compared to those of the magnetite particles, and also, generally had considerably high spin-charge densities, and were highly effective as magnetic drugs.

As described so far, according to the drug design method in the present embodiment, not only with the drugs comprising organic compounds but also with those comprising inorganic compounds, it is possible to analyze whether they are magnetic or not from molecular models thereof. Moreover, by examining drugs with high magnetic strength (that is, with high drug effects) in advance, it will become possible to design effective drugs with a considerably high efficiency.

The above-described cisplatin derivatives and the derivatives where platinum of the cisplatin derivatives was substituted by other metal elements are cis geometric isomers. Such cis geometric isomers are used as anticancer agents since they have higher effects at inhibiting the replication of DNA which is accompanied with the propagation of cancer cells than those in trans geometric isomers. However, according to the drug design method in the present embodiment, targeted drugs can be analyzed whether they are magnetic or not, not only when they are cis geometric isomers of anticancer agents or the like, but also when they are the metal complexes composed of trans geometric isomers or when they are other inorganic compounds. Therefore, it is also possible to design magnetic drugs comprising the metal complexes composed of trans geometric isomers, or other inorganic compounds.

Next is a description of a guidance system for guiding the abovementioned magnetic drug to a target site.

This guidance system may be any system as long as it generates a magnetic field, and various forms of systems can be considered. For example, as an example, application of magnetic resonance imaging (MRI) is considered, and the construction may be such that a magnetic field is irradiated to the human body and the magnetic field is controlled so as to guide the drug to the target site. Moreover, for example, a magnetic material such as a magnet may be adhered onto the skin surface of the target site. As a result, the drug that has reached the vicinity of the target site is guided to the target site, and stays specifically at the target site, causing no side effects to other normal cells. According to the above guidance system, it is possible to selectively and specifically guide a magnetic drug to the target site.

Furthermore, using the magnetism of the drugs administered in a body, it is also possible to examine the dynamics of the drugs in the body. More specifically, using a magnetic drug as a tracer, the dynamics of the drug in the body are examined by tracing the magnetism generated from the drug with a magnetic detector. With such a magnetic detector, it is possible to examine the dynamics of drugs in the body such as the time taken for the drugs to reach the target sites after being administered in the body and thus, the present invention can contribute to research and development of drugs.

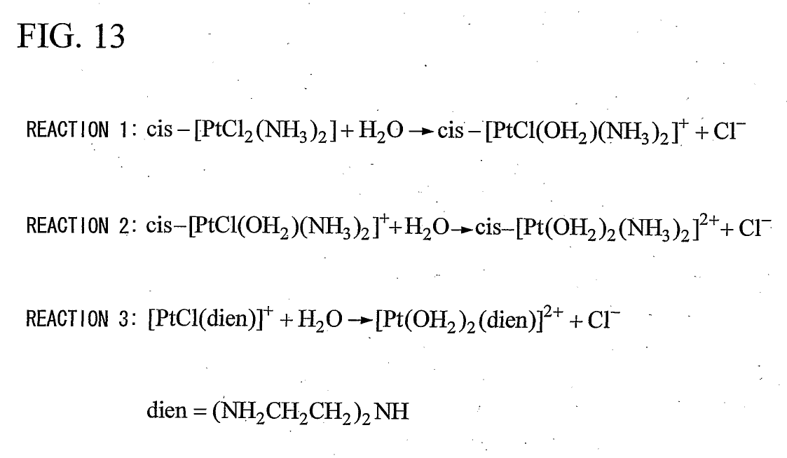

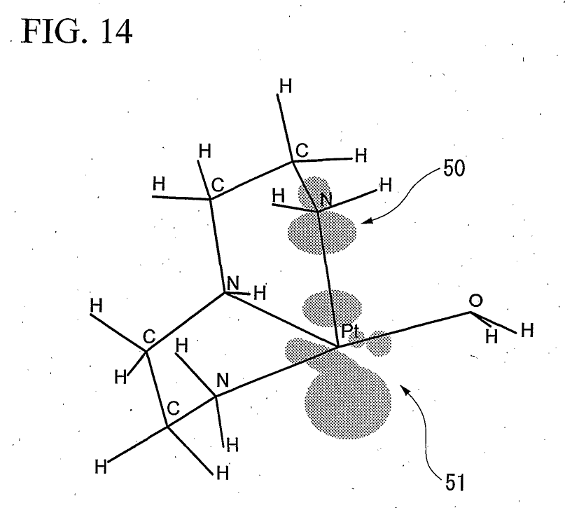

It is known that the cisplatin with a standard composition shown in FIG. 9 is hydrolyzed when administered in the body by the hydrolysis process represented by the reactions 1 to 3 shown in FIG. 13, and finally generates the hydrolysate of cisplatin [Pt(OH₂)₂(dien)]²⁺. As mentioned above, the cisplatin with a standard composition shown in FIG. 9 is not magnetic. However, the present inventors discovered that, based on the present drug design method, this hydrolysate of cisplatin [Pt(OH₂)₂(dien)]²⁺ is magnetic. FIG. 14 shows a three-dimensional molecular structure and spin-charge density distribution of the cisplatin hydrolysate [Pt(OH₂)₂(dien)]²⁺. As shown in this diagram, since the cisplatin hydrolysate [Pt(OH₂)₂(dien)]²⁺ has regions 50 and 51 with upward spin-charge densities, it is found to be a ferromagnetic body.

Therefore, even with the cisplatin with a standard composition, since it is magnetic after being administered in the body, it can be guided to the target site by the abovementioned guidance system and it is also possible to examine the dynamics thereof in the body with a magnetic detector.

While preferred embodiments of the invention have been described and illustrated above, it should be understood that these are exemplary of the invention and are not to be considered as limiting. Additions, omissions, substitutions, and other modifications can be made without departing from the spirit or scope of the present invention. Accordingly, the invention is not to be considered as being limited by the foregoing description, and is only limited by the scope of the appended claims.

1. A drug comprising an organic or inorganic compound, and which is made magnetic by modification of side chains and/or crosslinking between side chains.

2. A drug according to claim 1, wherein said organic compound is forskolin.

3. A drug according to claim 1, wherein said organic compound is a composition effective in treatment of male erectile dysfunction.

4. A drug according to claim 1, wherein said inorganic compound is a metal complex.

5. A drug according to claim 4, wherein said metal complex is a cis geometric isomer with anticancer properties.

6. A drug according to claim 5, wherein said cis geometric isomer is cisplatin.

7. A drug guidance system, wherein a drug according to claim 1 administered in a body is guided to a predetermined target site using the magnetism of the drug.

8. A magnetic detection system, wherein by detecting the magnetism of a drug according to claim 1 administered in a body, the dynamics of the drug in a body are detected.

9. A drug design method comprising: setting with respect to an organic or inorganic compound used as a drug, a molecular model having modified side chains and/or crosslinked side chains; determining whether or not said molecular model is magnetic, from a spin-charge density distribution obtained by a numerical calculation for said molecular model; and then designing said organic compound based on the molecular model that has been determined to be magnetic.

10. A drug design method according to claim 9, comprising determining whether said molecular model is ferromagnetic or ferrimagnetic, based on said spin-charge density distribution.

11. A drug design method according to claim 9, comprising determining the magnetic strength of said molecular model, based on said spin-charge density distribution.
